(19) United States
(12) Patent Application Publication
(10) Pub. No.: US 2021/0145645 A1
(43) Pub. Date: May 20, 2021

(54) OCCLUSION EFFECT REDUCING EARPLUG

(71) Applicant: TRITON SYSTEMS, INC., Chelmsford, MA (US)

(72) Inventor: Andrew DRAUDT, Arlington, MA (US)

(21) Appl. No.: 17/099,606

(22) Filed: Nov. 16, 2020

Related U.S. Application Data
(60) Provisional application No. 62/936,040, filed on Nov. 15, 2019.

(51) Int. Cl. A61F 11/10; G10K 11/162

(52) U.S. Cl. CPC A61F 11/10; G10K 11/162

(57) ABSTRACT

Occlusion effect reducing earplugs that have enhanced performance in reducing the occlusion effect are described. The occlusion effect reducing earplugs comprise a reservoir that form a region of high impedance mismatch with air. The reservoir can be a deep-seated reversible bi-stable reservoir that can be filled with a fluid. The occlusion effect reducing earplugs can also include a fitting plug that can serve to locate the balloon at depth and house one or more level-dependent filters, communication devices, and/or fluid filler bulbs.

### BACKGROUND

The Occlusion Effect is caused by intra-cranial sound passing into the occluded ear canal (see, FIG. 1, curved arrows pointing toward the ear canal). For the normal, un-occluded ear canal, the pressure in the canal is low. However, when the canal is partially or fully blocked at the outer end such as by an earplug or hearing aid, the sound pressure in the canal is orders of magnitude higher. This causes body-born sounds in the head (e.g., vocalization, breathing, footsteps) to couple more effectively into the ear canal and get to the eardrum, producing loud sound levels, especially at lower frequencies, that can be unpleasant for the user. For the case of level-dependent earplug, this extra noise can interfere with situational awareness and communication. Military personnel, for instance, often use this type of earplug since it blocks loud sounds like gun shots but allows normal level sounds to pass through. However, this type of earplug is plagued with the occlusion effect, and the extra sounds are a significant interference to communication and situational awareness.

Without being bound to any theory, an explanation for the occlusion effect can be obtained from the model shown in FIG. 2. The ear canal is modeled as a volume compliance, as the dimensions are much smaller than the wavelength at frequencies most evident in the occlusion effect (i.e., at about <1 kHz). On the medial end is the acoustic impedance of the tympanic membrane, and on the proximal end is either the radiation impedance of a tube radiating into open space or the impedance of the occluding element (e.g., earplug).

For open canal, the radiation impedance is low, and thus the pressure (voltage, in this circuit) is set by the radiation and source impedances. Since the radiation impedance is low, the pressure is low. However, for the case of the closed canal, the radiation impedance becomes the terminating impedance of the closed canal, which is high. The pressure in this case is set by the source impedance, canal volume, and eardrum impedance. The eardrum impedance is about 1000 times that of the open canal radiation impedance (at about 200 Hz). The canal volume compliance is even larger than that, behaving like a stiff spring. The pressure will thus be much higher than in the case of the open canal.

Using values for a foam earplug, an occluded canal length of about 10 or about 20 mm, and an intra-cranial pressure of about 1 Pa, the following parameters can be calculated: (1) a pressure in the canal of about 4.8e-3 Pa for a canal cavity of about 20 mm long; a pressure in the canal of about 2.9e-3 Pa for a canal cavity of about 10 mm long; and a pressure in the canal of about 8.4e-6 Pa for an open canal. The lower pressure in the shorter canal reflects a known aspect of the occlusion effect that deeper earplugs result in less pressure (Tufts et al., “Attenuation as a function of the canal length of custom-molded earplugs: A pilot study”. The Journal of the Acoustical Society of America, 133(6), pp. EL446-EL451 2013; and Lee, J. and Casali, J., Investigation of the auditory occlusion effect with implications for hearing protection and hearing aid design, Proc. Hum. Factors Ergon. Soc. Annu. Meet. 55 (2011) 1783-1787). In the above model, the canal length affects pressure via the acoustic volume and the source impedance. The source impedance is equal to ρC/area, where ρ and C are respectively the density and sound speed of the cartilage and the sound is applied over area, which scales with canal length. Thus, a shorter canal has higher source impedance, which results in a decrease in pressure. However, the magnitude of the impedance of the canal volume, ρC²/(volume*2πf), goes up with shorter canal length, thus tending to increase the pressure. In the above model, the change to the input impedance is the larger of the two effects, so the pressure drops. More sophisticated modeling can explain the loss in pressure for deeper plugs. Sound transmission across the medial canal walls is shown to be lower than the proximal portion, dominated by bone on the medial side (Kiessling et al., “Occlusion effect of earmolds with different venting systems”. Journal of the American Academy of Audiology, 16(4), pp. 237-249, 2005; and Stenfelt et al., “Factors contributing to bone conduction: The outer ear”. The Journal of the Acoustical Society of America, 113(2), pp. 902-913. 2013). The cause is presumably the higher impedance of bone as compared to cartilage. Stenfelt and Reinfeldt “A model of the occlusion effect with bone-conducted stimulation”. International journal of audiology, 46(10), pp. 595-608, 2007) showed this result using a more detailed lumped element model with separate sound inputs for the bone and cartilage portions of the canal. Brummund et al. (Three-dimensional finite element modeling of the human external ear: Simulation study of the bone conduction occlusion effect. The Journal of the Acoustical Society of America, 135(3), pp. 1433-1444. 2014; and An axisymmetric finite element model to study the earplug contribution to the bone conduction occlusion effect. Acta Acustica united with Acustica, 101(4), pp. 775-788, 2015) made sophisticated, 3D finite element models of the ear and surrounding tissue. With these models, they showed that significant energy passed through the earplugs themselves and into the remaining canal. They concluded: “an improved earplug design should aim at (i) reducing the power exchange between the ear canal wall and the earplug circumference and (ii) increas[ing] the power dissipation inside the earplug. Furthermore, in order for these improvements to be effective the earplug should occlude most of the cartilaginous ear canal.” Thus, Brummund et al. share the modern view that the main ways to defeat the occlusion effect are to use deep plugs to reduce transfer of sound from the cartilaginous canal walls to the air in the canal and to absorb that sound, or to include vents (Curran, A forgotten technique for resolving the occlusion effect. Starkey Innovations Magazine, 2(2), 2012). However, deep plugs present problems for comfort and practical insertion, and vents act as low radiation impedances to the outside, like the un-occluded case, but hence reduce the hearing protection to the wearer.

One way to reduce the occlusion effect is by using active noise cancellation (ANC). Notable commercially available products using ANC include the Bose® Hearphone™. However, this type of system is generally expensive, heavy, not robust, and not easy to use. The Bose product, for example, requires a relatively large battery worn around the neck and may not stand cold or wet environments.

The use of balloons in the ear canal has been disclosed (Amundsen, J., Next Generation Earplugs-A Deep Insert Solution, Master's thesis, NTNU, 2008; Lee and Casali, 2011; and Keady, J., Patent Appl. Pub. No. WO2009158624A1). Lee and Casali use an air-filled balloon, along with foam earplugs, to study the effect of insertion depth on the occlusion effect. Unsurprisingly, it was found that the balloon performed worst since it was filled with air and thus mimicked an air-filled cavity. Amundsen and Keady use balloons filled with water and gel respectively. These balloons start at the outer, distal end of the canal, and put pressure on the cartilaginous portion of the canal walls. They claim this extra pressure reduces the Occlusion Effect, but do not say why. Indeed, there is no scientific reason why this should be so. Instead the water balloon is a nearly perfect impedance match to the cartilage tissue (as compared with air) and thus will allow for efficient transfer of sound into the balloon, where it is then free to radiate into the inner portion of the canal.

Accordingly, there is a need for better earplugs that reduce the occlusion effect.

### SUMMARY

The invention discloses earplugs which reduce the Occlusion Effect caused by body-borne sound sources (such as breathing, speaking and talking) entering the ear canal, comprising a deeply inserted sound blocking element located at the inner proximal end of an ear canal near the ear drum, which serves to reflect sound from the outer region of the canal, comprising sound from the outside world as well as body sound coupled into the canal through the outer cartilaginous region of the canal. The reflection is caused by an acoustic impedance mis-match between the air in the outer canal and the material of the sound blocking element. Because the main source of body sound in the ear canal crosses into the ear canal from the cartilaginous region of the ear canal, and because this cartilaginous region occupies approximately the outer ½ of the ear canal, the sound blocking element resides approximately in the inner (proximal) ½ of the ear canal, in order to block the sound in the outer canal from passing to the ear drum.

In some embodiments, the sound blocking element is expandable, such as a fluid filled balloon or reversible bi-stable reservoir.

In some embodiments, the sound blocking element is a malleable material like foam rubber which can be squeezed to expand and press against the ear canal walls.

In some embodiments, the fluid in the balloon or reversible bi-stable reservoir is water.

In some embodiments, a tube passes through the sound blocking element which provides an efficient sound path from the ear drum to the plug, to enable the hearing of sound from outside the ear, if desired. Some existing products employ balloons which hold speakers close the ear drum. These resemble this invention, in that it is a deep balloon, however the material is often air, which does not have an impedance mismatch with air in the ear canal. This sound source will have its own occlusion effect, which such devices try to mitigate by having a hole in the balloon to let air and sound through. Those concepts and designs are not the same as those contemplated herein.

In some embodiments, the plug comprises one or more level-dependent filters.

In some embodiments, the plug comprises one or more communication elements.

### BRIEF DESCRIPTION OF THE DRAWINGS

The present disclosure is described with reference to the following figures, which are presented for the purpose of illustration only and are not intended to be limiting.

In the drawings:

FIG. 1 is a schematic representation of an Occlusion-Effect-Reducing Earplug (OERE) with a deep reversible bi-stable fluid reservoir in accordance with aspects of the present disclosures;

FIG. 2 is a schematic representation of a design of an OERE with a deep reversible bi-stable fluid reservoir in accordance with aspects of the present disclosures;

FIG. 3A is an image depicting a setup with an OERE in accordance with aspects of the present disclosures;

FIG. 3B is an image depicting an ear-mold in accordance with aspects of the present disclosures;

FIG. 3C is an image depicting a bone exciter in accordance with aspects of the present disclosures; and

FIG. 4 is a plurality of graphs presenting the occlusion effect over different frequencies for earplug, earplug with water balloon, and water balloon in accordance with aspects of the present disclosures.

FIG. 5A depicts an exemplary device inserted in the ear canal with a fluid reservoir pushed into position such that the reservoir engages the ear canal wall.

FIG. 5B depicts an exemplary device with an expanded fluid reservoir, which does not engage the ear canal for entry and removal.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person in the art will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

Various terms are used herein consistent with their common meanings in the art. The following terms are defined below for clarity.

It must also be noted that as used herein and in the appended claims, the singular forms “a”, “an”, and “the” include plural reference unless the context clearly dictates otherwise. Thus, for example, reference to “an earplug” is a reference to “one or more earplug” and equivalents thereof known to those skilled in the art, and so forth.

As used herein, the term “about” means plus or minus 10% of the numerical value of the number with which it is being used. Therefore, about 50% means in the range of 45%-55%.

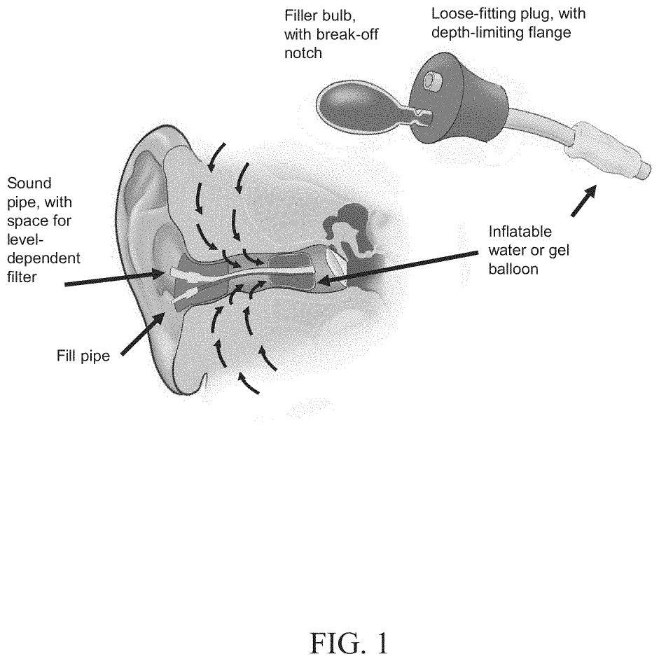

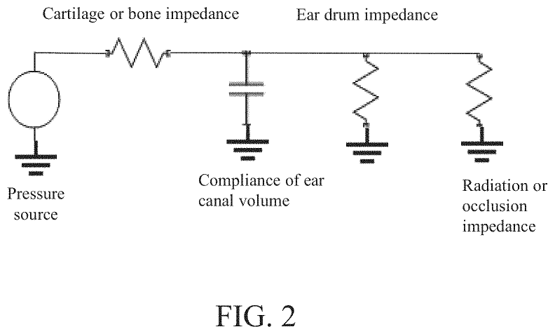

In some embodiments, an occlusion effect reducing earplug (OERE) is disclosed. With reference to FIG. 1, the OERE can include a sound blocking element such as, but not limited to, a fluid reservoir (e.g., a water balloon) that surrounds a hollow pipe. The fluid reservoir can be a deep-seated reversible bi-stable fluid reservoir. The sound blocking element can block not only the ambient sound but also the main source of occlusion-effect sound that is coming from the outer regions of the ear canal. One of ordinary skill in the art would appreciate that the sound blocking element is more comfortable in the ear canal than other devices such as elastic solid devices. The OERE can also include a cylindrical and fitting plug that can serve to locate the reservoir at depth and house one or more level-dependent filters, communication devices, and mechanisms to pump fluid into and out of the sound blocking element if appropriate. The OERE can also include a central tube that can conduct sound from the outside to enable near-normal hearing with or without a level dependent filter. The OERE can also include a level-dependent filter. The level-dependent filter can allow for natural hearing or electronic communications elements such as one or more receptors, emitters, electronics, and/or batteries.

In some embodiments, the sound blocking element is located at about 1 mm from the eardrum, at about 2 mm from the eardrum, about 3 mm from the eardrum, about 4 mm from the eardrum, about 5 mm from the eardrum, about 6 mm from the eardrum, about 7 mm from the eardrum, about 8 mm from the eardrum, at about 9 mm from the eardrum, at about 10 mm from the eardrum, at about 11 mm from the eardrum, at about 12 mm from the eardrum, at about 13 mm from the eardrum, at about 14 mm from the eardrum, at about 15 mm from the eardrum, at about 16 mm from the eardrum, at about 17 mm from the eardrum, at about 18 mm from the eardrum, at about 19 mm from the eardrum, at about 20 mm from the eardrum, at about 21 mm from the eardrum, at about 22 mm from the eardrum, at about 23 mm from the eardrum, at about 24 mm from the eardrum, or at about 25 mm from the eardrum.

In some embodiments, a reusable or disposable OERE is disclosed. In the disposable design, the user can, for example, insert a plug comprising a fluid reservoir (e.g. a balloon) and squeeze a filler bulb to fill the balloon, break off the bulb, and/or have the fluid remain inside the balloon via a one-way valve. In this case, a custom ear-mold may not be needed and the plug may not have to be tight-fitting since the balloon itself can provide attenuation of exterior sound. The fluid reservoir may also be filled by a pumping mechanism, syringe, or other internal or external device.

In other embodiments, the sound blocking element can be a malleable material such as foam or other elastic material. In such embodiments, the malleable material can be compressed and held in place by a sleeve until the device is properly positioned. Once in place, the sleeve can be retracted, and the malleable material allowed to expand to seal the ear canal. In some embodiments, the malleable material may be covered with a skin material. Similarly, axial compression can also be used to achieve a Poisson effect expansion. Axial compression may be achieve by a wire or rod that extends through the elastic element and is pulled out against a sleeve (like a bike brake) via various mechanisms such as a push button or lever.

In some embodiments, a reusable or disposable OERE is disclosed. In the reusable design, a mechanism can plump fluid into and out of the balloon.

Some embodiments may further include a removal device such as a pull tab or string to facilitate removal of the device from the ear canal.

Similarly, some embodiments may further include a tube and valve to allow pressure release during insertion and/or removal to minimize baratrauma.

Without being bound to any theory, the majority of input energy to the occlusion effect comes from the outer, cartilaginous region of the canal, and this can be prevented from reaching the eardrum with a region of high impedance mismatch with air (e.g., a fluid) inserted in the boney region of the canal via the fluid reservoir (e.g., a balloon filled with a fluid such as water). There may still be a sound source from this boney region but it may be less intense than the outer region and may contribute less energy due to a smaller area through which to radiate. However, the smaller cavity volume thus created near the eardrum may have a large acoustic impedance, which may increase the energy transmission efficiency from this boney region.

The earplugs disclosed herein can be used in many applications. For instance, some of the earplugs disclosed herein can be used in dosimetry. The noise exposure of workers is a topic of importance for health, science, protection, and legal reasons. It is desired to measure the dose directly in the user's ear, often behind hearing protection. Earplugs generally have high variability in protection due to how well they fit in the ear. Many scientific methods and inventions have attempted to account for this variability. The occlusion effect also has a large impact on the user's sound exposure by adding sound that would not have been present without an earplug. Some of the earplugs disclosed herein can solve both of these problems by giving a less variable and a more constant and long-lasting fit and by partially or completely removing the occlusion effect. The earplugs disclosed herein can provide the sound level right at the eardrum, thus eliminating not only the occlusion effect but also the resonances in the ear canal, which are highly variable between individuals. The sound at the eardrum can be measured through the central tube, which has known constant acoustic affect that can be accounted for.

Furthermore, some of the earplugs disclosed herein can be used for hearing testing. Hearing tests often use in-ear sound sources. The actual sound delivered to the eardrum from these may be influenced by the individual ear canal shape and level of occlusion of the device. The earplugs disclosed herein can deliver the sound directly to the eardrum, without effects from the canal or occlusion.

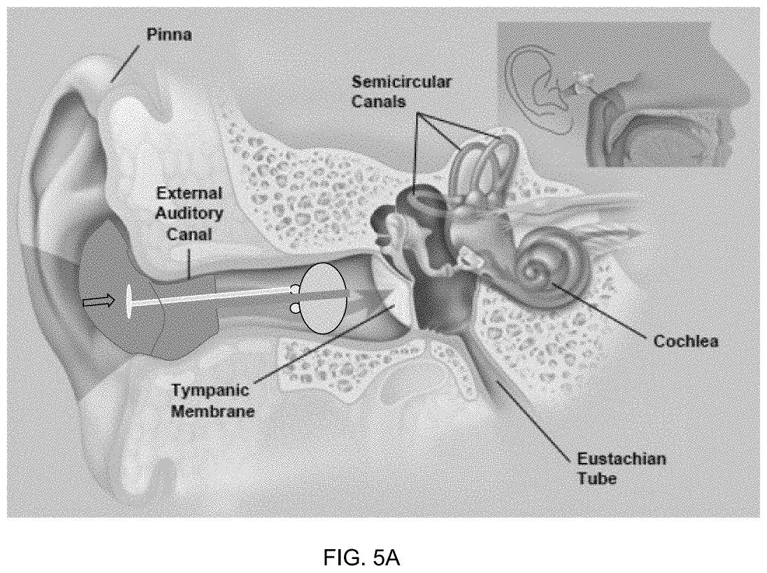

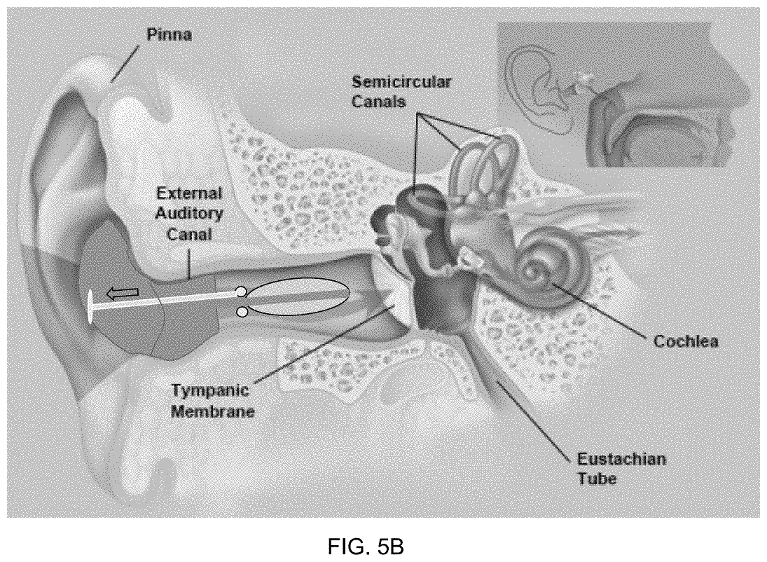

Furthermore, some of the earplugs disclosed herein can be used in hearing aids. Some hearing aids are design to fit deeply in the ear canal, much like some of the earplugs disclosed herein. Reasons for this deep fit include the reduction of the occlusion effect and the invisible and long term use. The fitting and sizing of the hearing aid and the insertion process may be unpleasant for the user. Only a portion of people can tolerate them. Some of the earplugs disclosed herein can be used in conjunction with hearing aid electronics, to deliver the same results but provide more comfort, easier insertion, and less customization for the user. FIGS. 5A and 5B depict a schematic view of an exemplary ear plug in accordance with some embodiments The Figs. Depict a pumping mechanism that can move fluid into and out of a fluid reservoir, such as a balloon. A movable cylinder or ring (e.g. o-ring) can move a long shallow balloon down to the end of the device, causing the fluid to make a wider balloon and form a seal with the ear canal.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

### Occlusion Effect Reducing Earplug

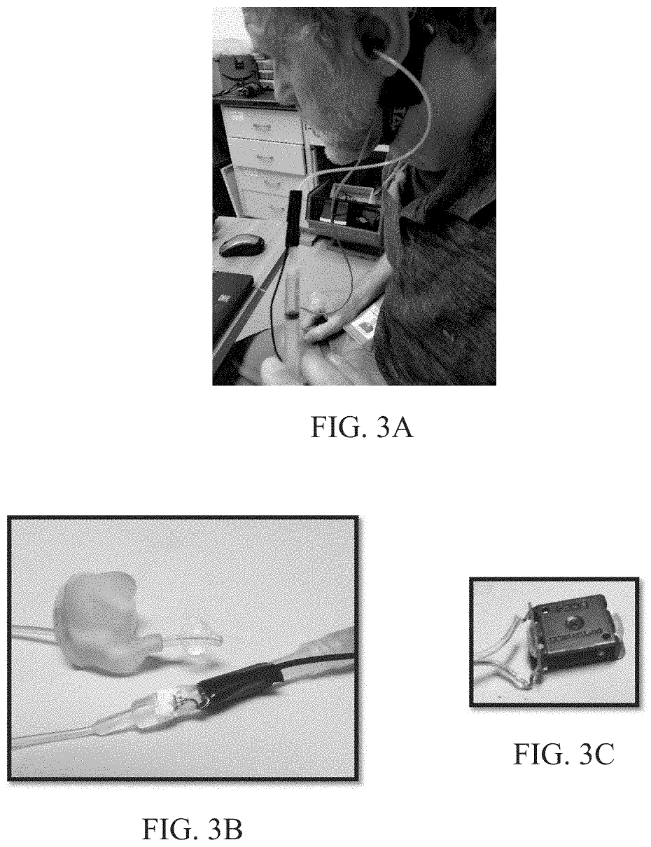

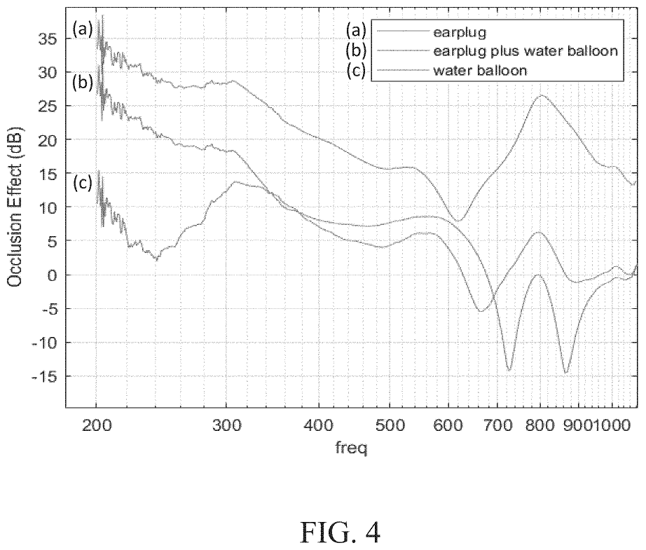

An occlusion effect reducing earplug (OERE) was fabricated. The OERE is illustrated in FIGS. 3A, 3B, and 3C. The OERE consisted of a Foley catheter (Medline, 6 Fr., 1.5 mL) inserted through a hole cut into a hard plastic ear-mold (see, FIGS. 3A and 3B). The ear-mold was 3D printed from a CAD file provided by a Lantos ear scanning system. A miniature microphone (Knowles EM 23047) was sealed over the end of the central pipe of the catheter, thus acting as a probe mic inserted near the eardrum (tube effects did not affect this frequency range). A bone exciter transducer (see, FIG. 3C) was strapped to the mastoid process, and issued a chirp signal (200 Hz-1 kHz). The sound was recorded by the probe mic, for the cases of:
(1) Open canal: catheter inserted, no ear mold, and balloon not inflated;
(2) Ear-mold inserted, and balloon not inflated (full occlusion effect);
(3) Ear-mold inserted, balloon inflated with water (occlusion effect reduced);
(4) Ear-mold removed, balloon still in place.
   The open canal measurement was subtracted from the rest of the measurements, to yield a plot of the occlusion effect (see, FIG. 4). These results show greater than 10 dB reduction in occlusion effect for most of the frequency band.

### ABSTRACT

Occlusion effect reducing earplugs are described that have enhanced performance in reducing the occlusion effect from intracranial sound sources such as breathing, speaking, and coughing. The occlusion effect reducing earplug comprises a deep sound-blocking element that form a region of high impedance mismatch with air. The blocking element can be a deep-seated reversible bi-stable reservoir that can be filled with a fluid. The occlusion effect reducing earplugs can also include a fitting plug that can serve to locate the balloon at depth and house one or more level-dependent filters, communication devices, and/or fluid filler bulbs.

What is claimed:

1. An earplug comprising:
a sound blocking element located at a distal end of an ear canal; and
a plug located at a proximal end of the ear canal, wherein the plug is connected to the sound blocking element;
wherein the sound blocking element is designed and configured for deep placement within the ear canal.

2. The earplug of claim 1, wherein the sound blocking element has a high impedance mismatch compared to the air in the ear canal.

3. The earplug of claim 1, wherein the sound blocking element is a fluid reservoir or a malleable material capable of expanding to seal the ear canal.

4. The earplug of claim 1, wherein the sound blocking element is a fluid reservoir in the form of a balloon, and the earplug further comprises a pump or filler bulb to move fluid into the balloon.

5. The earplug of claim 1, wherein the fluid reservoir is a reversible bi-stable reservoir.

6. The earplug of claim 1, wherein the sound blocking element is located at about 5 mm from an eardrum.

7. The earplug of claim 4, where the fluid filler bulb is filled with a high impedance mismatch fluid when compared to the air in the ear canal.

8. The earplug of claim 7, wherein the high impedance mismatch fluid is water.

9. The earplug of claim 1, wherein the plug comprises one or more level-dependent filters.

10. The earplug of claim 1, wherein the plug comprises one or more communication elements.

11. The earplug of claim 1, wherein the sound blocking element is a malleable material.

12. The earplug of claim 11, wherein the sound blocking element comprises a malleable material.

13. The earplug of claim 12, wherein the malleable material is a foam or other elastic material.

14. The earplug of claim 13, wherein the malleable material is compressed within a sleeve until the device is properly positioned; the sleeve being retractable to allow the malleable material to expand to seal the ear canal.
